(19) United States
(12) Patent Application Publication
(10) Pub. No.: US 2026/0275294 A1
(43) Pub. Date: Sep. 17, 2026

(54) COMPOSITIONS AND METHODS FOR PRESERVATION OF ISOLATED MITOCHONDRIA

(71) Applicant: The Sallie A Burdine Breast Foundation, Baton Rouge, LA (US)

(72) Inventors: Xianpeng Jiang, Baton Rouge, LA (US); Catherine Baucom, Baton Rouge, LA (US); Brent M. Segal, Pembroke, MA (US)

(21) Appl. No.: 19/166,287

(22) PCT Filed: Mar. 28, 2024
(86) PCT No.: PCT/US24/22014

§ 371 Date: Sep. 17, 2025

Related U.S. Application Data
(60) Provisional application No. 63/455,397, filed on Mar. 29, 2023.
(60) Provisional application No. 63/604,044, filed on Nov. 29, 2023.

(51) Int. Cl. C12N 5/0775; A61K 9/50; A61K 35/00; A61K 35/12; A61K 35/741; A61K 45/06; C07K 14/705; C12N 5/00; C12N 5/077

(52) U.S. Cl. CPC C12N 5/0662; A61K 9/5068; A61K 35/12; A61K 35/741; A61K 45/06; C07K 14/70596; C12N 5/0656; C12N 5/526; C12N 5/562; A61K 2035/115; C12N 2533/52

(57) ABSTRACT

Described herein are compositions and methods for the preservation of the membrane potential of isolated mitochondria, such mitochondria to be used in mitochondrial organelle transplantation methods and compositions. For example, the present disclosure provides for methods and compositions for preservation of isolated mitochondria using extracellular vesicles (EVs).

### CROSS-REFERENCE TO RELATED APPLICATIONS

This application claims priority to and the benefit of U.S. Provisional Patent Application No. 63/455,397, filed on Mar. 29, 2023, and U.S. Provisional Patent Application No. 63/604,044, filed on Nov. 29, 2023, the disclosures of which are incorporated by reference in their entireties.

### FIELD

The subject matter described herein relates to compositions and methods for the preservation of isolated mitochondria. In certain embodiments, the mitochondria thusly preserved are used for mitochondrial organelle transplantation in a subject.

### BACKGROUND

Mitochondria are organelles found within most eukaryotic cells. They serve an important function in cellular respiration and the generation of adenosine triphosphate (ATP). Mitochondrial organelle transplantation (MOT) was developed as a strategy for treatment of mitochondrial dysfunction or injury. In recent years, mitochondrial organelle transplantation (MOT) has shone a new light on therapeutic intervention that benefits neuronal survival and regeneration for neurodegenerative diseases, stroke, and CNS injury. However, current MOT procedures need isolation of fresh mitochondria. Isolated mitochondria which are stored for long periods of time may experience functional declines, making them less suitable for use in MOT. Accordingly, there exists a need for improved mitochondrial organelle transplantation methods.

### SUMMARY

The present disclosure provides for methods and compositions for preservation of isolated mitochondria using extracellular vesicles (EVs). Membranous EVs, including exosomes, microvesicles and apoptotic bodies (ApoBDs), facilitate intercellular communication following their release from donor cells and subsequent internalization into recipient cells. Additionally, as described herein, EVs can also be used in combination with isolated mitochondria to facilitate longer term preservation of mitochondria for use in mitochondrial organelle transplantation (MOT). For example, as described herein, mitochondria mixed with EVs showed enhanced functionality after cold storage over a period of multiple days. Functional measures including mitochondrial membrane potential (MMP) and mitochondrial ATP were shown to be higher than mitochondria stored under similar conditions without EVs. Moreover, EVs can be frozen at low temperatures (e.g., −20° C. or lower) for long periods of time with minimal losses experienced by cellular components stored within the EVs including isolated mitochondria.

In one aspect, the invention is directed to a composition (e.g., a pharmaceutical composition) (e.g., for the preservation of mitochondrial membrane potential of isolated mitochondria for use in mitochondrial organelle transplantation), the composition comprising: isolated mitochondria; and extracellular vesicles (EVs).

In some embodiments, the extracellular vesicles comprise one or more members selected from the group consisting of (i), (ii), and (iii) as follows: (i) microvesicles (MVs), exosomes, and apoptotic bodies; (ii) microvesicles (MVs); and (iii) apoptotic bodies. In some embodiments, the extracellular vesicles comprise microvesicles (MVs). In some embodiments, the microvesicles (MVs) range from about 100 nm to about 1 micrometer in diameter. In some embodiments, the microvesicles (MVs) comprise cytosolic and plasma membrane associated proteins.

In some embodiments, the extracellular vesicles (EVs) comprise microvesicles (MVs). In some embodiments, the microvesicles (MVs) range from about 30 nm to about 150 nm in diameter. In some embodiments, the microvesicles (MVs) are formed by an endosomal route.

In some embodiments, the extracellular vesicles comprise apoptotic bodies. In some embodiments, the apoptotic bodies range from about 50 nm up to about 5 micrometers in diameter. In some embodiments, the apoptotic bodies comprise one or more members selected from the group consisting of intact organelles, chromatin, and glycosylated proteins.

In some embodiments, the extracellular vesicles comprise extracellular vesicles of mesenchymal stromal cells (imEVs). In some embodiments, the imEVs are obtained from mesenchymal stromal cells cultured on a substrate (e.g., a cell culture plate) coated with laminin (e.g., laminin-521).

In some embodiments, the isolated mitochondria comprises mitochondria isolated from fibroblasts (e.g., fibroblasts of a human donor, e.g., primary fibroblasts).

In some embodiments, the isolated mitochondria comprises mitochondria isolated from mesenchymal stromal cells (MSCs) (e.g., MSCs of a human donor).

In some embodiments, the composition comprises a mixture of mitochondria and EVs in a ratio from about 1:50 (mitochondria:EVs, in vol.) to about 50:1 (mitochondria:EVs, in vol.) [e.g., wherein the ratio is from about 2:1 to about 50:1, or wherein the ratio is from about 5:1 to about 15:1, or wherein the ratio is about 9:1]. In some embodiments, the composition comprises a mixture of mitochondria and EVs in a ratio from about 5:1 (mitochondria:EVs, in vol.) to about 15:1 (mitochondria:EVs, in vol.).

In some embodiments, the composition is or comprises a mitochondrial storing buffer (e.g., a mitochondrial isolation buffer composition comprising components as described in the above-referenced International (PCT) Patent Application No. PCT/US2020/047359, incorporated herein by reference, e.g., with the addition of the extracellular vesicles) (e.g., wherein the mitochondrial storing buffer has a potassium ion concentration safe for administration to humans). In some embodiments, the mitochondrial storing buffer comprises: a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose, e.g., sucrose at a concentration of about 240 mM); an agent that acts as a membrane stabilizer and/or oxygen radical scavenger and/or binder of Ca2+ and/or binder of free fatty acid (e.g., bovine serum albumin, BSA); and a serine protease inhibitor (e.g., phenylmethylsulfonyl fluoride (PMSF), also called phenylmethane sulfonyl fluoride).

In some embodiments, the composition is or comprises a mitochondrial isolation buffer composition (e.g., a mitochondrial isolation buffer composition comprising components as described in the above-referenced International (PCT) Patent Application No. PCT/US2020/047359, incorporated herein by reference, e.g., with the addition of the extracellular vesicles). In some embodiments, the mitochondrial isolation buffer composition comprises: a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(O-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca2+ and/or binder of free fatty acid (e.g., bovine serum albumin, BSA); and a serine protease inhibitor (e.g., phenylmethylsulfonyl fluoride (PMSF), also called phenylmethane sulfonyl fluoride).

In some embodiments, the composition comprises a pharmaceutically acceptable carrier.

In some embodiments, the mitochondria accumulate within structures formed by the EVs in the composition.

In some embodiments, the composition does not comprise an antibiotic (e.g., such that the isolated mitochondria are not exposed to an antibiotic and/or such that a subject is not administered an antibiotic).

In some embodiments, the EVs are positive for one, two, or all three of the following surface markers: CD63, CD81, and TSG101.

In some embodiments, the EVs are negative for the surface marker Calnexin.

In some embodiments, the composition can be stored (e.g., preserved) for a time up to about 5 days (e.g., up to about 24 hours, e.g., up to about 48 hours) (e.g., without a significant loss of mitochondrial membrane potential (MMP)) (e.g., while preserving/maintaining mitochondrial adenosine triphosphate (ATP) content).

In some embodiments, the mitochondrial ATP content of the isolated mitochondria (e.g., after storage) is at least 80% of the mitochondrial ATP content of freshly isolated mitochondria (e.g., at least 70%, at least 60%, at least 50%, at least 40%).

In some embodiments, the mitochondrial membrane potential (MMP) of the isolated mitochondria (e.g., after storage) is substantially the same as the MMP of freshly isolated mitochondria (e.g., at least 95% of the MMP of freshly isolated mitochondria, e.g., at least 90%, at least 80%, at least 70%, at least 60%, at least 50%).

In some embodiments, the composition can be stored for at least about 48 hours or more (e.g., at least one hour, at least 2 hours, at least 3 hours, at least about 6 hours, e.g., at least about 12 hours, e.g., at least about 24 hours, e.g., at least about 48 hours or more).

In some embodiments, the composition can be stored at a reduced temperature. In some embodiments, the reduced temperature is a temperature below 15° C. (e.g., e.g., below 10° C., e.g., at a temperature within a range from about 0° C. to about 15° C., e.g., from about 1° C. to about 10° C., e.g., from about 2° C. to about 6° C.). In some embodiments, the reduced temperature is a temperature at or below 0° C. (e.g., at or below −10° C., at or below −20° C.) (e.g., stored using liquid nitrogen) (e.g., stored on dry ice).

In another aspect, the invention is directed to a method comprising using a composition (e.g., as described herein) comprising isolated mitochondria and extracellular vesicles to improve preservation of mitochondrial membrane potential (MMP) of the isolated mitochondria (e.g., using the extracellular vesicles to improve preservation of MMP of the isolated mitochondria and/or to improve preservation/retention of mitochondrial adenosine triphosphate (ATP) content), wherein the isolated mitochondria are used in any of the mitochondrial organelle transplantation methods and/or compositions and/or kits described in the above-referenced International (PCT) Patent Application No. PCT/US2020/047359 or U.S. Provisional Patent Application No. 63/405,336, both incorporated herein by reference.

In another aspect, the invention is directed to method for transplantation of mitochondria in a human subject, said method comprising: storing the mitochondria isolated from a donor in a storage composition comprising extracellular vesicles (EVs); and after the storing step, administering to said subject a mitochondrial composition comprising said stored mitochondria.

In some embodiments, the method comprises storing the storage composition at a reduced temperature (e.g., without a significant loss of mitochondrial membrane potential (MMP)) (e.g., while preserving/maintaining mitochondrial adenosine triphosphate (ATP) content). In some embodiments, the reduced temperature is a temperature below 15° C. (e.g., e.g., below 10° C., e.g., at a temperature within a range from about 0° C. to about 15° C., e.g., from about 1° C. to about 10° C., e.g., from about 2° C. to about 6° C.). In some embodiments, the reduced temperature is a temperature at or below 0° C. (e.g., at or below −10° C., at or below −20° C.) (e.g., stored using liquid nitrogen) (e.g., stored on dry ice).

In some embodiments, the method comprises storing the storage composition for at least about 48 hours or more (e.g., at least one hour, at least 2 hours, at least 3 hours, at least about 6 hours, e.g., at least about 12 hours, e.g., at least about 24 hours, e.g., at least about 48 hours or more) prior to administering the composition.

In some embodiments, the extracellular vesicles comprise one or more members selected from the group consisting of (i), (ii), and (iii) as follows: (i) microvesicles (MVs) (e.g., ranging from about 100 nm to about 1 micrometer in diameter, e.g., comprising cytosolic and plasma membrane associated proteins), exosomes, and apoptotic bodies; (ii) microvesicles (MVs) (e.g., ranging from about 30 nm to about 150 nm in diameter, e.g., formed by an endosomal route); and (iii) apoptotic bodies (e.g., ranging from about 50 nm up to about 5 micrometers in diameter, e.g., comprising intact organelles and/or chromatin and/or glycosylated proteins).

In some embodiments, the extracellular vesicles comprise extracellular vesicles of mesenchymal stromal cells (imEVs).

In some embodiments, the subject has a neurodegenerative disease or other condition associated with mitochondrial dysfunction (e.g., as described herein).

In some embodiments, the mitochondrial composition further comprises a mitochondrial storing buffer having a potassium ion concentration safe for administration to humans.

In some embodiments, the administering step comprises parenterally administering at least one-unit dose of said mitochondrial composition to said subject.

In some embodiments, the administering step comprises both intramuscular injection and intravenous injection of said mitochondrial composition to said subject.

In some embodiments, the mitochondrial composition administered to the subject does not comprise an antibiotic.

In some embodiments, the method comprises administering to the subject an iron-chelating agent (e.g., desferrioxamine or deferasirox).

In some embodiments, the method comprises administering to the subject an antioxidant and/or a probiotic.

In another aspect, the invention is directed to a kit comprising a mitochondrial composition in a unit dosage (e.g., a therapeutically effective amount) effective to treat a neurodegenerative disease or other condition associated with mitochondrial dysfunction in a subject, said mitochondrial composition comprising: isolated mitochondria; and extracellular vesicles (EVs).

In some embodiments, the kit further comprises instructions for optimizing the dose and/or frequency and/or route of administration of the composition.

In another aspect, the invention is directed to a method of storing a composition (e.g., as described herein) comprising isolated mitochondria and extracellular vesicles to improve preservation of MMP of the isolated mitochondria and/or to improve preservation/retention of mitochondrial adenosine triphosphate (ATP) content, the method comprising storing said composition at a reduced temperature.

In some embodiments, the reduced temperature is a temperature below 15° C. (e.g., below 10° C., e.g., at a temperature within a range from about 0° C. to about 15° C., e.g., from about 1° C. to about 10° C., e.g., from about 2° C. to about 6° C.).

In some embodiments, the reduced temperature is a temperature at or below 0° C. (e.g., at or below −10° C., at or below −20° C.) (e.g., stored using liquid nitrogen) (e.g., stored on dry ice).

In some embodiments, the method comprises storing the composition for a time up to about 24 hours (e.g., up to about 48 hours, e.g., up to about 5 days).

In some embodiments, the method comprises storing the composition for at least one hour (e.g., at least 2 hours, at least 3 hours, at least about 6 hours, e.g., at least about 12 hours, e.g., at least about 24 hours, e.g., at least about 48 hours, e.g., at least about 5 days).

In some embodiments, the mitochondrial ATP content of the isolated mitochondria (e.g., after storage) is at least 80% of the mitochondrial ATP content of freshly isolated mitochondria (e.g., at least 70%, at least 60%, at least 50%, at least 40%).

In some embodiments, the mitochondrial membrane potential (MMP) of the isolated mitochondria (e.g., after storage) is substantially the same as the MMP of freshly isolated mitochondria (e.g., at least 95% of the MMP of freshly isolated mitochondria, e.g., at least 90%, at least 80%, at least 70%, at least 60%, at least 50%).

Any two or more of the features described in this specification, including in this summary section, may be combined to form implementations not specifically or explicitly described in this specification.

### Definitions

A or An: The articles “a” and “an” are used herein to refer to one or to more than one (i.e., to at least one) of the grammatical object of the article. By way of example, “an element” refers to one element or more than one element.

Administration: As used herein, the term “administration” typically refers to the administration of a composition to a subject or system, for example to achieve delivery of an agent that is, is included in, or is otherwise delivered by, the composition. Non-limiting examples of administration include oral administration; parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Agent: As used herein, the term “agent” refers to an entity (e.g., for example, a cell, a component of a cell such as mitochondria or other organelle, a small molecule, a peptide, a polypeptide, a nucleic acid, a lipid, a polysaccharide, a complex, a combination, a mixture, a system, or a phenomenon such as heat, electric current, electric field, magnetic force, magnetic field, etc.).

Amelioration: As used herein, the term “amelioration” refers to the prevention, reduction, palliation, or improvement of a state of a subject. Amelioration includes, but does not require, complete recovery or complete prevention of a disease, disorder or condition.

Antibiotic: As used herein, the term “antibiotic” refers to an antibacterial substance such as penicillin, gentamicin, streptomycin, cephalosporin, ciprofloxacin, or the like, that is used to treat or prevent infections by killing or inhibiting the growth of bacterial in or on the body, that is administered orally, topically, or by injection, and that is isolated from cultures of certain microorganisms (such as fungi) or is of semi-synthetic or synthetic origin.

Biological Sample: As used herein, the term “biological sample” typically refers to a sample obtained or derived from a biological source (e.g., a tissue or organism or cell culture) of interest, as described herein. In some embodiments, e.g., as set forth herein, a biological source is or includes an organism, such as an animal or human. In some embodiments, e.g., as set forth herein, a biological sample is or includes biological tissue or fluid. In some embodiments, e.g., as set forth herein, a biological sample can be or include cells, tissue (e.g., skin tissue, muscle, or other tissue), or bodily fluid. In some embodiments, e.g., as set forth herein, a biological sample can be or include blood, blood cells, cell-free DNA, free floating nucleic acids, ascites, biopsy samples, surgical specimens, cell-containing body fluids, sputum, saliva, feces, urine, cerebrospinal fluid, peritoneal fluid, pleural fluid, lymph, gynecological fluids, secretions, excretions, skin swabs, vaginal swabs, oral swabs, nasal swabs, washings or lavages such as a ductal lavages or bronchioalveolar lavages, aspirates, scrapings, bone marrow. In some embodiments, e.g., as set forth herein, a biological sample is or includes cells obtained from a single subject or from a plurality of subjects. A sample can be a “primary sample” obtained directly from a biological source or can be a “processed sample.” A biological sample can also be referred to as a “sample.”

Improved, increased, or reduced: As used herein, these terms, or grammatically comparable comparative terms, indicate values that are relative to a comparable reference measurement. For example, in some embodiments, e.g., as set forth herein, an assessed value achieved with an agent of interest may be “improved” relative to that obtained with a comparable reference agent or with no agent. Alternatively or additionally, in some embodiments, e.g., as set forth herein, an assessed value in a subject or system of interest may be “improved” relative to that obtained in the same subject or system under different conditions or at a different point in time (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, e.g., as set forth herein, comparative terms refer to statistically relevant differences (e.g., differences of a prevalence and/or magnitude sufficient to achieve statistical relevance). Those of skill in the art will be aware, or will readily be able to determine, in a given context, a degree and/or prevalence of difference that is required or sufficient to achieve such statistical significance.

Isolated: As used herein, “isolated” refers to a substance and/or entity (e.g., including one or more mitochondria) that has been (a) separated from at least some of the components with which it was associated when initially produced (whether in nature, in a subject such as a donor, and/or in an experimental setting), and/or (b) designed, produced, prepared, and/or manufactured by the hand of man. Isolated substances and/or entities may be separated from at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated substances and/or entities are at least about 80%, about 85%, about 90%, about 91%, about 92%, about 93%, about 94%, about 95%, about 96%, about 97%, about 98%, about 99%, or more than about 99% pure. As used herein, a substance and/or entity is “pure” if it is substantially free of other components. In some embodiments, as will be understood by those skilled in the art, a substance and/or entity may still be considered “isolated” or “pure” after having been combined with certain other components such as, for example, one or more carriers or excipients (e.g., buffer, solvent, water, etc.); in such embodiments, percent isolation or purity of the substance and/or entity is calculated without including such carriers or excipients. To give but one example, in some embodiments, mitochondria that occur in nature can be referred to as “isolated” when, (a) they are present in a composition that does not include some or all of the components with which they were associated in nature, e.g., in a donor from which they were derived; (b) they are substantially free of other organelles of a donor organism from which they were derived; (c) they are present in a cell or system that differs from the donor organism from which they were derived. Thus, for instance, mitochondria removed from a donor for transplantation into a second different subject can be referred to as “isolated.”

Neurodegenerative disease: As used herein, the term “neurodegenerative disease” (also referred to as “degenerative nerve disease”) is an umbrella term for conditions which primarily affect the neurons in the human brain. In certain instances, neurodegenerative disease is characterized by a progressive loss of neurons associated with deposition of proteins showing altered physicochemical properties in the brain and/or in peripheral organs. Neurodegenerative diseases include, for example, Alzheimer's disease (AD), amyotrophic lateral sclerosis (ALS), Batten disease, dementia, fatal familial insomnia, Friedreich's ataxia, Huntington's disease (HD), Lewy body dementia (LBD), Parkinson's disease (PD) and PD-related disorders, mitochondrial disorders, motor neuron diseases (MND), multiple sclerosis (MS), muscular dystrophy (MD), prion diseases, spinal muscular atrophy (SMA), spinocerebellar ataxia (SCA), and others.

Pharmaceutical composition: As used herein, the term “pharmaceutical composition” refers to a composition in which an active agent is provided together with one or more pharmaceutically acceptable carriers. In some embodiments, e.g., as set forth herein, the active agent is present in a unit dose amount appropriate for administration to a subject, e.g., in a therapeutic regimen that shows a statistically significant probability of achieving a predetermined therapeutic effect when administered to a relevant population. In some embodiments, e.g., as set forth herein, a pharmaceutical composition can be formulated for administration in a particular form (e.g., in a solid form or a liquid form), and/or can be specifically adapted for, for example: oral administration (for example, as a drenche (aqueous or non-aqueous solutions or suspensions), tablet, capsule, bolus, powder, granule, paste, etc., which can be formulated specifically for example for buccal, sublingual, or systemic absorption); parenteral administration (for example, by subcutaneous, intramuscular, intravenous or epidural injection as, for example, a sterile solution or suspension, or sustained-release formulation, etc.); topical application (for example, as a cream, ointment, patch or spray applied for example to skin, lungs, or oral cavity); intravaginal or intrarectal administration (for example, as a pessary, suppository, cream, or foam); ocular administration; nasal or pulmonary administration, etc.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation and/or modifies bioavailability of an agent, e.g., a pharmaceutical agent. Some examples of materials which can serve as pharmaceutically-acceptable carriers include: sugars, such as lactose, glucose and sucrose; starches, such as com starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Prevent or prevention: The terms “prevent” and “prevention,” as used herein in connection with the occurrence of a disease, disorder, or condition, refers to reducing the risk of developing the disease, disorder, or condition; delaying onset of the disease, disorder, or condition; delaying onset of one or more characteristics or symptoms of the disease, disorder, or condition; and/or to reducing the frequency and/or severity of one or more characteristics or symptoms of the disease, disorder, or condition. Prevention can refer to prevention in a particular subject or to a statistical impact on a population of subjects. Prevention can be considered complete when onset of a disease, disorder, or condition has been delayed for a predefined period of time.

Prognosis: As used herein, the term “prognosis” refers to determining the qualitative or quantitative probability of at least one possible future outcome or event. As used herein, a prognosis can be a determination of the likely course of a disease, disorder, or condition such as cancer in a subject, a determination regarding the life expectancy of a subject, or a determination regarding response to therapy, e.g., to a particular therapy.

Reference: As used herein describes a standard or control relative to which a comparison is performed. For example, in some embodiments, e.g., as set forth herein, an agent, subject, animal, individual, population, sample, sequence, or value of interest is compared with a reference or control agent, subject, animal, individual, population, sample, sequence, or value. In some embodiments, e.g., as set forth herein, a reference or characteristic thereof is tested and/or determined substantially simultaneously with the testing or determination of the characteristic in a sample of interest. In some embodiments, e.g., as set forth herein, a reference is a historical reference, optionally embodied in a tangible medium. Typically, as would be understood by those of skill in the art, a reference is determined or characterized under comparable conditions or circumstances to those under assessment, e.g., with regard to a sample. Those skilled in the art will appreciate when sufficient similarities are present to justify reliance on and/or comparison to a particular possible reference or control.

Sample: As used herein, the term “sample” typically refers to an aliquot of material obtained or derived from a source of interest. In some embodiments, e.g., as set forth herein, a source of interest is a biological or environmental source. In some embodiments, e.g., as set forth herein, a sample is a “primary sample” obtained directly from a source of interest. In some embodiments, e.g., as set forth herein, as will be clear from context, the term “sample” refers to a preparation that is obtained by processing of a primary sample (e.g., by removing one or more components of and/or by adding one or more agents to a primary sample).

Susceptible to: An individual who is “susceptible to” a disease, disorder, or condition is at risk for developing the disease, disorder, or condition. In some embodiments, e.g., as set forth herein, an individual who is susceptible to a disease, disorder, or condition does not display any symptoms of the disease, disorder, or condition. In some embodiments, e.g., as set forth herein, an individual who is susceptible to a disease, disorder, or condition has not been diagnosed with the disease, disorder, and/or condition. In some embodiments, e.g., as set forth herein, an individual who is susceptible to a disease, disorder, or condition is an individual who has been exposed to conditions associated with, or presents a biomarker status associated with, development of the disease, disorder, or condition. In some embodiments, e.g., as set forth herein, a risk of developing a disease, disorder, and/or condition is a population-based risk (e.g., family members of individuals suffering from the disease, disorder, or condition).

Subject: As used herein, the term “subject” or “patient” refers to an organism, typically a mammal (e.g., a human). In some embodiments, e.g., as set forth herein, a subject is suffering from a disease, disorder or condition. In some embodiments, e.g., as set forth herein, a subject is susceptible to a disease, disorder, or condition. In some embodiments, e.g., as set forth herein, a subject displays one or more symptoms or characteristics of a disease, disorder or condition. In some embodiments, e.g., as set forth herein, a subject is not suffering from a disease, disorder or condition. In some embodiments, e.g., as set forth herein, a subject does not display any symptom or characteristic of a disease, disorder, or condition. In some embodiments, e.g., as set forth herein, a subject is someone with one or more features characteristic of susceptibility to or risk of a disease, disorder, or condition. In some embodiments, e.g., as set forth herein, a subject is a patient. In some embodiments, e.g., as set forth herein, a subject is an individual to whom diagnosis has been performed and/or to whom therapy has been administered. In some instances, e.g., as set forth herein, a human subject can be interchangeably referred to as an “individual.”

Therapeutic agent, pharmaceutical agent, and active agent: As used herein, the terms “therapeutic agent”, “pharmaceutical agent”, and “active agent” are interchangeable, and each refers to any agent that elicits a desired pharmacological effect when administered to a subject. In some embodiments, e.g., as set forth herein, an agent is considered to be a therapeutic agent if it demonstrates a statistically significant effect across an appropriate population. In some embodiments, e.g., as set forth herein, the appropriate population can be a population of model organisms or a human population. In some embodiments, e.g., as set forth herein, an appropriate population can be defined by various criteria, such as a certain age group, gender, genetic background, preexisting clinical conditions, etc. In some embodiments, e.g., as set forth herein, a therapeutic agent is a substance that can be used for treatment of a disease, disorder, or condition. In some embodiments, e.g., as set forth herein, a therapeutic agent is an agent that has been or is required to be approved by a government agency before it can be marketed for administration to humans. In some embodiments, e.g., as set forth herein, a therapeutic agent is an agent for which a medical prescription is required for administration to humans.

Therapeutically effective amount: As used herein, the term “therapeutically effective amount” refers to an amount that produces a desired effect for which it is administered. In some embodiments, e.g., as set forth herein, the term refers to an amount that is sufficient, when administered to a population suffering from or susceptible to a disease, disorder, or condition, in accordance with a therapeutic dosing regimen, to treat the disease, disorder, or condition. Those of ordinary skill in the art will appreciate that the term therapeutically effective amount does not in fact require successful treatment be achieved in a particular individual. Rather, a therapeutically effective amount can be an amount that provides a particular desired pharmacological response in a significant number of subjects when administered to individuals in need of such treatment. In some embodiments, e.g., as set forth herein, reference to a therapeutically effective amount can be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent can be formulated and/or administered in a single dose. In some embodiments, e.g., as set forth herein, a therapeutically effective agent can be formulated and/or administered in a plurality of doses, for example, as part of a multi-dose dosing regimen.

Treatment: As used herein, the term “treatment” (also “treat” or “treating”) refers to administration of a therapy that partially or completely alleviates, ameliorates, relieves, inhibits, delays onset of, halts progression of, slows progression of, reverses progression of, reduces severity of, and/or reduces incidence of one or more symptoms, features, and/or causes of a particular disease, disorder, or condition, or is administered for the purpose of achieving any such result. In some embodiments, e.g., as set forth herein, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, e.g., as set forth herein, treatment can be of a subject who has been diagnosed as suffering from the relevant disease, disorder, and/or condition. In some embodiments, e.g., as set forth herein, treatment can be of a subject known to have one or more susceptibility factors that are statistically correlated with increased risk of development of the relevant disease, disorder, or condition. In some embodiments, treatment is of a cancer.

Unit dose: As used herein, the term “unit dose” refers to an amount administered as a single dose and/or in a physically discrete unit of a pharmaceutical composition. In many embodiments, e.g., as set forth herein, a unit dose contains a predetermined quantity of an active agent. In some embodiments, e.g., as set forth herein, a unit dose contains an entire single dose of the agent. In some embodiments, e.g., as set forth herein, more than one-unit dose is administered to achieve a total single dose. In some embodiments, e.g., as set forth herein, administration of multiple unit doses is required, or expected to be required, in order to achieve an intended effect. A unit dose can be, for example, a volume of liquid (e.g., comprising an pharmaceutically acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic agent(s). For example, acceptable carriers (e.g., pharmaceutically acceptable carriers), diluents, stabilizers, buffers, preservatives, etc., can be included. It will be appreciated by those skilled in the art, in many embodiments, e.g., as set forth herein, a total appropriate daily dosage of a particular therapeutic agent can comprise a portion, or a plurality, of unit doses, and can be decided, for example, by a medical practitioner within the scope of sound medical judgment. In some embodiments, e.g., as set forth herein, the specific effective dose level for any particular subject or organism can depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

### BRIEF DESCRIPTION OF THE DRAWINGS

The foregoing and other objects, aspects, features, and advantages of the present disclosure will become more apparent and better understood by referring to the following description taken in conjunction with the accompanying drawings, in which:

FIG. 1, panels A-C show characterizations of the BM-MSC derived EVs using (panel A) nanoparticle tracking analysis (NanoSight 3000) of the EVs, (panel B) transmission electron microscopy of the EVs and (panel C) western-blot analysis of the EVs (EV) and the BM-MSC cell lysates (Cell lysate) for surface markers of EVs, according to an illustrative embodiment.

FIG. 2, panels A and B show JC-1-stained mitochondria in imEVs, according to an illustrative embodiment.

FIG. 3 shows a Western Blot for genes in imEVs, according to an illustrative embodiment.

FIG. 4A shows JC-1 stained mitochondria without imEVs, according to an illustrative embodiment.

FIG. 4B shows JC-1 stained mitochondria mixed with imEVs, according to an illustrative embodiment.

FIG. 5 shows a graph of MMP of isolated mitochondria and isolated mitochondria with imEVs, according to an illustrative embodiment.

FIG. 6 shows a graph of mitochondrial ATP content of isolated mitochondria and isolated mitochondria with imEVs, according to an illustrative embodiment.

FIG. 7, panels A and B show exemplary images of isolated mitochondria in mixtures of extracellular vesicles (EVs), according to an illustrative embodiment.

FIG. 8, panels A and B show exemplary images of isolated mitochondria in mixtures of extracellular vesicles (EVs), according to an illustrative embodiment.

FIG. 9 shows images of isolated mitochondria (“Mito”) and mitochondria in mixtures of extracellular vesicles (“Mito+EVs”) 2 days post-isolation, according to an illustrative embodiment.

FIG. 10 shows images of isolated mitochondria (“Mito”) and mitochondria in mixtures of extracellular vesicles (“Mito+EVs”) 5 days post-isolation, according to an illustrative embodiment.

FIG. 11 is a flow diagram of an exemplary method, according to an illustrative embodiment.

### DETAILED DESCRIPTION

Throughout the description, where compositions, articles, devices, and systems are described as having, including, or comprising specific components, or where processes and methods are described as having, including, or comprising specific steps, it is contemplated that, additionally, there are compositions, articles, devices, and systems of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication is not an admission that the publication serves as prior art with respect to any of the claims presented herein.

Headers are provided for the convenience of the reader—the presence and/or placement of a header is not intended to limit the scope of the subject matter described herein.

Incorporated herein is U.S. Provisional Patent Application No. 63/405,336, filed Sep. 9, 2022, entitled, “Compositions and Methods for Treatment of Traumatic Brain Injury (TBI), for Example, Mild Traumatic Brain Injury (mTBI)”, which describes compositions and methods involving mitochondrial organelle transplantation for use in the treatment of traumatic brain injury (TBI), e.g., mild traumatic brain injury (mTBI), in a subject. Also incorporated herein is International (PCT) Patent Application No. PCT/US2020/047359, filed Aug. 21, 2020, and published as International Publication No. WO 2021/141637, which describes compositions and methods for treatment of amyotrophic lateral sclerosis (ALS); other neurodegenerative diseases (ND) such as Alzheimer's disease, Parkinson's disease, muscular dystrophy; and other mitochondrial disorders. In particular, described therein are experimental examples demonstrating mitochondrial organelle transplantation (MOT™) for the treatment of NDs such as ALS. This application claims the benefit of U.S. patent application Ser. No. 16/937,388, filed on Jul. 23, 2020, and U.S. Provisional Patent Application No. 62/958,592, filed Jan. 8, 2020. The contents of each of the above-referenced patent applications are incorporated by reference herein in their entirety. Moreover, all publications mentioned herein are incorporated by reference herein in their entirety.

Described herein are compositions, methods, and kits for the preservation of the membrane potential of isolated mitochondria, such mitochondria to be used in the mitochondrial organelle transplantation methods and compositions described in the above publications.

Mixing isolated mitochondria with extracellular vesicles (EVs) and/or nanoparticles, or incorporating isolated mitochondria within these structures, may enhance the useful lifetime of such isolated mitochondria for mitochondrial organelle transplantation. The improved stability may be useful in the packaging and/or storing of therapeutic compositions comprising such isolated mitochondria.

In eukaryotic cells, mitochondria are well known as the powerhouse which generates ATP by oxidative phosphorylation (OXPHOS). They also play an important role in synthesis of iron-sulfur clusters and heme, β-oxidation of fatty acids, the urea cycle, and homeostasis of calcium, iron and reactive oxygen species (ROS). Mitochondrial dysfunction plays an important role in many diseases including, but not limited to, cardiovascular disease, metabolic disease, neurodegenerative disease. Traditional drugs or genes have difficulty entering specific sub-compartments of mitochondria. Moreover, the diverse nature of gene mutations among patients makes it impossible to develop one drug to ameliorate mitochondrial dysfunction.

In recent years, mitochondrial organelle trans-plantation (MOT) has shone a new light on therapeutic interventions that benefit neuronal survival and regeneration for neurodegenerative diseases, stroke, and CNS injury. For example, McCully et al. reported the first clinical trial of mitochondrial transplantation therapy. They performed an autologous mitochondrial transplantation for myocardial ischemia-reperfusion injury of pediatric patients who required extracorporeal membrane oxygenation (ECMO). In another example, Elliott et al. reported a case study where MOT significantly improved leg muscle strength and recovered sensory sensations in a deteriorated ALS patient.

However, isolated mitochondria loses significant activity when stored on ice for more than 1 hour, thus a rapid operation is essential during clinical trials. Cold storage and cryopreservation of mitochondria have not been successful for long term storage, leading to a decrease in respiratory capacity of mitochondria and damage to mitochondrial membrane structures over time. Thus, preservation of mitochondria is a significant challenge for MOT.

Membranous extracellular vesicles (EVs), including exosomes, microvesicles and apoptotic bodies (ApoBDs), facilitate intercellular communication following their release from donor cells and subsequent internalization into recipient cells. EVs can encapsulate small portions of the subjacent cytosol and create a heterogeneous population of phospholipid-walled vesicles. ApoBDs are the largest EVs in size and contain a wide variety of cellular components: micronuclei, chroma-tin remnants, cytosol portions, degraded proteins, DNA fragments, or even intact organelles. MSC-EV-mediated mitochondrial transfer from the EVs to injured alveolar epithelia and endothelia has been reported. In the acute respiratory distress syndrome (ARDS) models, MSC-EVs improve alveolar-capillary barrier properties through restoration of mitochondrial functions at least partially via mitochondrial transfer. EVs can be stored at −20° C. without losing the efficacy of inner cellular components including mitochondria. Accordingly, EVs could be used to preserve the function of cellular components and enhance the stability of isolated mitochondria.

### A. MITOCHONDRIA

In eukaryotic cells, mitochondria are known as the powerhouse, which generates adenosine triphosphate (ATP), by oxidative phosphorylation (OXPHOS). They also play an important role in synthesis of iron-sulfur clusters and heme, β-oxidation of fatty acids, homeostasis of calcium, iron and reactive oxygen species (ROS). Mitochondria are of particular importance in neurons. Neurons have high metabolic requirements—the brain consumes 20% of the body's resting ATP production despite being only 2% of its mass. Moreover, mitochondria are essential calcium buffering organelles in neurons that modulate local calcium dynamics, for example, regulate neurotransmitter release. Neurons are long-lived cells that persist throughout the lifespan of the individual and as such are more susceptible to the accumulating damage arising from mitochondrial dysfunction. Severe mitochondrial dysfunction comes in many forms, including defective OXPHOS, excessive ROS, impaired calcium buffering capacity, and defective mitochondrial dynamics

In eukaryotic cells, mitochondria generate ATP by oxidative phosphorylation (OXPHOS) in the presence of oxygen. Mitochondria also play an important role in synthesis of iron-sulfur (Fe—S) clusters, β-oxidation of fatty acids, synthesis of heme prosthetic groups, the urea cycle, as well as homeostasis of calcium, iron and reactive oxygen species (ROS). Mitochondria are highly dynamic organelles which frequently fuse and divide. Mitochondrial fusion/fission allow segregation of damaged mitochondria, mitophagy to remove damaged mitochondria, and ultimately cell death if the damage is too severe. In addition, mitochondria can transfer between cells. Cells may be able to obtain functional mitochondria from other cells in order to satisfy their bioenergetics and biosynthetic needs. Without wishing to be bound to any particular theory, possible mechanisms include tunneling nanotubes, extracellular vesicles, and partial or complete cell fusion.

Mitochondrial dysfunction contributes to many diseases such as neurodegenerative disease, cardiac disease, and cancer. Mitochondrial dysfunction broadly includes states in which mitochondria of a cell, tissue, organism, or sample thereof, are characterized by (1) a decreased rate, amount, or efficiency of ATP production: (2) a decreased mitochondrial membrane potential; (3) a decreased number or concentration of mitochondria; and/or (4) an increased rate or amount of ROS production, relative to a reference. In some embodiments, a reference is a measurement or value representative of a healthy subject (e.g., a comparable subject with typical mitochondrial function and/or without a diagnosed medical condition known to impact mitochondrial function). In some embodiments, a reference is a measurement or value representative of a population of healthy subjects. In some embodiments, a reference is a measurement or value representative of the subject at an earlier time. Methods and techniques for measuring mitochondrial ATP production, mitochondrial membrane potential, number or concentration of mitochondria, and/or ROS production are known in the art.

Mitochondrial dysfunction has been documented in amyotrophic lateral sclerosis (ALS), Alzheimer's disease (AD), and Parkinson's disease (PD), for example. Mitochondria are essential for neural function because neurons highly depend on aerobic OXPHOS in mitochondria for their energetic needs. Defective mitochondrial respiration and ATP production in neurons result in neural dysfunction and degeneration. Mitochondria also produce ROS. If oxidative stress of ROS overwhelms the antioxidative defense most from superoxide dismutase (SOD), catalase (CAT) and glutathione peroxidase (GPX), ROS causes protein, lipid and DNA damage of neurons. In addition, overload of mitochondrial calcium and iron impairs ATP production and structures of mitochondria and neurons.

Mitochondria are highly present in cardiac cells due to the increased energy demands of such cells. Mitochondrial dysfunction is associated with the development of numerous cardiac diseases such as atherosclerosis, ischemia-reperfusion injury, hypertension, cardiac hypertrophy and heart failure.

Defects in mitochondrial function have also been linked to tumorigenesis. For example, it has been observed that cancer cells have an increase in glycolysis and lactate production in the presence of oxygen without an increase in OXPHOS, known as the “Warburg Effect”. Many cancers have mitochondrial defects and dysfunction. Glycolytic inhibitors have been found to suppress tumor growth in animal models and clinical trials.

In certain embodiments, mitochondria can be isolated from cells (herein, referred to herein as “isolated mitochondria”). In certain embodiments, isolated mitochondria comprises mitochondria isolated from a human donor. In certain embodiments, isolated mitochondria are obtained from fibroblasts (e.g., fibroblasts of a human donor, e.g., primary fibroblasts of a human donor) and/or mesenchymal stromal cells (MSCs) (e.g., MSCs of a human donor).

### B. EXTRACELLULAR VESICLES & STORAGE WITH IMEVS

Membranous extracellular vesicles (EVs) are membranous structures that facilitate intercellular communication following their release from donor cells and subsequent internalization into recipient cells.

Three subtypes of extracellular vesicles (EVs) include the following: (i) microvesicles (MVs), which generally range from about 100 nm to about 1 micrometer in diameter and primarily contain cytosolic and plasma membrane associated proteins; (ii) exosomes, which generally range from about 30 nm to about 150 nm in diameter and are formed by an endosomal route; and (iii) apoptotic bodies, which generally range from about 50 nm up to about 5 micrometers in diameter and generally contain intact organelles, chromatin, and small amounts of glycosylated proteins.

In certain embodiments described herein, extracellular vesicles comprise EVs of mesenchymal stromal cells (imEVs). In certain embodiments, imEVs are obtained from MSCs cultured on a substrate (e.g., a cell culture plate, e.g., a glass cell culture plate, a plastic cell culture plate) coated with a protein (e.g., an extracellular matrix protein). In certain embodiments, cells are cultured on a substrate coated with laminin (e.g., laminin-521). In certain embodiments, EVs are positive for one, two, or all three of the following surface markers: CD63, CD81, and TSG101. In certain embodiments, EVs are negative for the marker Calnexin.

In certain embodiments, a composition described herein comprises a mixture of mitochondria and EVs. In certain embodiments, mitochondria accumulate within structures formed by EVs in a composition. In certain embodiments, a mixture of mitochondria and EVs has a ratio from about 1:50 (mitochondria:Evs, in vol.) to about 50:1 (mitochondria:Evs, in vol.). In certain embodiments, the ratio is from about 2:1 to about 50:1. In certain embodiments, the ratio is from about 5:1 to about 15:1. In certain embodiments, the ratio is about 9:1.

In certain embodiments, compositions comprising isolated mitochondria and extracellular vesicles improve preservation of mitochondrial membrane potential (MMP) of the isolated mitochondria and/or improve preservation/retention of mitochondrial adenosine triphosphate (ATP) content, wherein the isolated mitochondrial are used in any of the mitochondrial organelle transplantation methods and/or compositions and/or kits described in the above-referenced International (PCT) Patent Application No. PCT/US2020/047359 or U.S. Provisional Patent Application No. 63/405,336, both incorporated herein by reference.

In certain embodiments, compositions comprising EVs and mitochondria are stored at a reduced temperature (e.g., at a temperature below 15° C., e.g., below 10° C., e.g., at a temperature within a range from about 0° C. to about 15° C., e.g., from about 1° C. to about 10° C., e.g., from about 2° C. to about 6° C.). In certain embodiments, compositions comprising EVs and mitochondria are stored for a time up to about 24 hours, e.g., up to about 48 hours, e.g., up to about 5 days). In certain embodiments, compositions comprising EVs and mitochondria are stored for at least 1 hour (e.g., at least 2 hours, at least 3 hours, at least about 6 hours, at least about 12 hours, at least about 24 hours, at least about 48 hours, at least about 5 days).

In certain embodiments, compositions described herein are stored at or below 0° C. (e.g., at or below −10° C., at or below −20° C.). For example, compositions can be stored using liquid nitrogen or on dry ice.

In some embodiments, storage of mitochondria-EV compositions is performed such that the mitochondrial ATP content of the isolated mitochondria after storage is preserved/maintained. In some embodiments, isolated mitochondria after storage has at least 80% of the mitochondrial ATP content of freshly isolated mitochondria is preserved/maintained after storage (e.g., at least 70%, at least 60%, at least 50%).

In some embodiments, storage of mitochondria-EV compositions is performed such that the mitochondrial membrane potential (MMP) of the isolated mitochondria after storage is substantially the same (or similar to) the MMP of freshly isolated mitochondria. For example, in some embodiments, at least 95% of the MMP of freshly isolated mitochondria is preserved/maintained after storage (e.g., at least 90%, at least 80%, at least 70%, at least 60%, at least 50%).

### C. MITOCHONDRIAL ISOLATION AND STORING BUFFERS

In certain embodiments, methods and compositions described herein utilize isolation and storing buffers (e.g., respiration buffers) for mitochondria. In certain embodiments, mitochondrial isolation and storing buffers used are described in International (PCT) Patent Application No. PCT/US2020/047359, filed Aug. 21, 2020, which is incorporated by reference in its entirety. In certain embodiments, a composition is or includes a mitochondrial storing buffer having a potassium ion concentration safe for administration to humans (e.g., wherein said mitochondrial storing buffer comprises a pharmaceutically acceptable carrier) (e.g., a mitochondrial storing buffer composition comprising components as described in the above-referenced International (PCT) Patent Application No. PCT/US2020/047359, incorporated herein by reference, e.g., with the addition of the extracellular vesicles). In certain embodiments, a composition is or includes a mitochondrial isolation buffer composition (e.g., a mitochondrial isolation buffer composition comprising components as described in the above-referenced International (PCT) Patent Application No. PCT/US2020/047359, incorporated herein by reference, e.g., with the addition of the extracellular vesicles). In certain embodiments, mitochondria accumulate within structures formed by EVs in a composition. In certain embodiments, a composition does not comprise an antibiotic (e.g., such that the isolated mitochondria are not exposed to an antibiotic and/or such that the subject is not administered an antibiotic).

In certain embodiments, compositions described herein comprise isolated mitochondria and nanoparticles (e.g., nanoparticles that are not EVs).

In certain embodiments, an isolation buffer comprises a buffering agent [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)]; a chelating agent [e.g., ethylene glycol-bis(β-aminoethyl ether)-N,N,N′,N′-tetraacetic acid (EGTA) or salt thereof, e.g., K-EGTA)]; a sugar (e.g., sucrose); an agent that acts as a membrane stabilizer and/or oxygen radical scavenger and/or binder of Ca²⁺ and/or binder of free fatty acid (e.g., bovine serum albumin, BSA); and a serine protease inhibitor (e.g., phenylmethylsulfonyl fluoride (PMFS), also called phenylmethane sulfonyl fluoride). In certain embodiments, the composition further comprises isolated donor mitochondria, e.g., fibroblast mitochondria.

In certain embodiments, a mitochondrial isolation buffer is comprised of 300 mM sucrose, 10 mM K-HEPES, 1 mM K-EGTA, 0.1% BSA and 0.25 mM PMSF (Sigma Aldrich, St Louis, MO, USA). In certain embodiments, the osmolarity of the buffer is about 325 mOsm. In certain embodiments, the concentration of potassium ion is 11 mM. Bovine serum albumin (BSA) is a membrane stabilizer, oxygen radical scavenger, and binds Ca2+ and free fatty acids. Phenylmethylsulfonyl fluoride (PMSF), also called phenylmethane sulfonyl fluoride, is a serine protease inhibitor used in the preparation of cell lysates. Lysosomes are organelles that contain digestive enzymes which digest excess or worn out organelles. During the procedure of cell homogenization (e.g., to obtain mitochondria), some lysosomes may be damaged and release the digestive enzymes to the cell lysate. In certain embodiments, in order to prevent the damage of mitochondria from the digestive enzymes, PMSF is included in the isolation buffer. In certain embodiments, the isolation buffer does not contain antibiotics.

In certain embodiments, a mitochondrial storage buffer is administered to a subject and/or used to maintain isolated mitochondria stably in solution. In certain embodiments, a storage buffer comprises one or more buffering agents [e.g., a zwitterionic sulfonic acid buffering agent, e.g., 4-(2-hydroxyethyl)-1-piperazineethanesulfonic acid (HEPES) or salt thereof, e.g., HEPES potassium salt, (K-HEPES)][e.g., monopotassium phosphate (KH₂PO₄)]; a source of magnesium ion [e.g., magnesium chloride (MgCl₂)]; a chelating agent [e.g., ethylene glycol-bis(O-aminoethyl ether)-N,N,N′,N′-tetraacetic acid (EGTA) or salt thereof, e.g., K-EGTA)]; a sugar (e.g., sucrose); an antioxidant [e.g., taurine]; a cytoprotective agent that binds to calcium ion [e.g., lactobionate or salt thereof, e.g., K-lactobionate]; and an agent that acts as a membrane stabilizer and/or oxygen radical scavenger and/or binder of Ca²⁺ and/or binder of free fatty acid (e.g., bovine serum albumin, BSA). In certain embodiments, the composition further comprises isolated donor mitochondria (e.g., fibroblast mitochondria).

In certain embodiments, a mitochondrial storing buffer is comprised of 240 mM sucrose, 2 mM KH₂PO₄, 3 mM MgCl₂, 10 mM K-HEPES, 20 mM Taurine, 1 mM K-EGTA, 0.1% BSA and 15 mM K-lactobionate (Sigma Aldrich, St Louis, MO, USA). Taurine acts as an antioxidant that scavenges free radical species generated by mitochondria, and is also involved to membrane stabilization, osmoregulation and ion channel regulation. Lactobionate has cytoprotective property and prevents mitochondrial swelling. Lactobionate also binds to calcium ion with high affinity and acts as a calcium chelator. In certain embodiments, the osmolarity of a mitochondrial storing buffer is about 325 mOsm. In certain embodiments, a mitochondrial storing buffer contains about 28 mM potassium ion. In certain embodiments, buffers are sterilized by filtering (e.g., through 0.22 μm filter), aliquoted to small vials and stored at −80° C.

In certain embodiments (e.g., clinical uses discussed herein), mitochondria in storing buffer are administered (e.g., intramuscularly, intravenously, subcutaneously) (e.g., both intramuscularly and intravenously) to a human subject. High potassium ion concentrations are dangerous for injection to humans (e.g., 91 mM potassium ion concentration). In certain embodiments, a reduced concentration of all K⁺ salts is used in a mitochondrial storing buffer (e.g., 2 mM KH₂PO₄, 10 mM K-HEPES, and 15 mM K-lactobionate). In certain embodiments, the final concentration of K⁺ in a storing buffer solution used is similar to clinical intravenous solution with potassium chloride (e.g., about 28 mEq) (e.g., from 20 mEq to 40 mEq). To keep the osmolarity of a storing buffer at a desired level, the concentration of sucrose can be increased in a storing buffer. In certain embodiments, a storing buffer is not administered along with and/or does not include antibiotics.

### D. REFERENCES

Murphy, E., Ardehali, H., Balaban, R. S., DiLisa, F., Doml, G. W., et al. (2016) Mitochondrial Function, Biology, and Role in Disease. Circulation Research, 118, 1960-1991. https://doi.org/10.1161/RES.0000000000000104.
Braymer, J. J. and Lill, R. (2017) Iron-Sulfur Cluster Biogenesis and Trafficking in Mitochondria. Journal of Biological Chemistry, 292, 12754-12763. https://doi.org/10.1074/jbc.R117.787101
Herst, P. M., Rowe, M. R., Carson, G. M. and Berridge, M. V. (2017) Functional Mitochondria in Health and Disease. Frontiers in Endocrinology, 9, 1-16. https://doi.org/10.3389/fendo.2017.00296.
Smith, E. F., Shaw, P. J. and De Vos, K. J. (2019) The Role of Mitochondria in Amyotrophic Lateral Sclerosis. Neuroscience Letters, 710, Article ID: 132933. https://doi.org/10.1016/j.neulet.2017.06.052
Federico, A., Cardaioli, E., Pozzo, P. D., Formichi, P., Gallus, G. N. and Radi, E. (2012) Mitochondria, Oxidative Stress and Neurodegeneration. Journal of the Neurological Sciences, 322, 254-262. https://doi.org/10.1016/j.jns.2012.05.030
Elliott, R. and Jiang, X. (2020) Neurodegeneration and Mitochondria Organelle Transplantation: “A Technology That Proof of Principle Suggest Is Ready for Prime Time”. Neuroscience and Medicine, 11, 108-118. https://doi.org/10.4236/nm.2020.114013
Norat, P., Soldozy, S., Sokolowski, J. D., Gorick, C. M., et al. (2020) Mitochondrial Dysfunction in Neurological Disorders: Exploring Mitochondrial Transplantation. npj Regenerative Medicine, 5, Article No. 22. https://doi.org/10.1038/s41536-020-00107-x
Emani, S. M., Piekarski, B. L., Harrild, D., Del Nido, P. J. and McCully, J. D. (2017) Autologous Mitochondrial Transplantation for Dysfunction after Ischemia-Reperfusion Injury. The Journal of Thoracic and Cardiovascular Surgery, 154, 286-289. https://doi.org/10.1016/j.jtcvs.2017.02.018
McCully, J. D., Cowan, D. B., Emani, S. M. and Del Nido, P. J. (2017) Mitochondrial Transplantation: From Animal Models to Clinical Use in Humans. Mitochondrion, 34, 127-134. https://doi.org/10.1016/j.mito.2017.03.004
Yamada, Y., Momo Ito, M., Arai, M., Hibino, M., Tsujioka, T. and Harashima, H. (2020) Challenges in Promoting Mitochondrial Transplantation Therapy. International Journal of Molecular Sciences, 21, Article 6365. https://doi.org/10.3390/ijms2t176365
Murphy, D. E., de Jong, O. G., Brouwer, M., Wood, M. J., Lavieu, G., Schiffelers, R. M. and Pieter Vader, P. (2019) Extracellular Vesicle-Based Therapeutics: Natural versus Engineered Targeting and Trafficking. Experimental & Molecular Medicine, 51, 1-12. https://doi.org/10.1038/st2276-019-0223-5
Battistelli, M. and Falcieri, E. (2020). Apoptotic Bodies: Particular Extracellular Vesicles Involved in Intercellular Communication. Biology, 9, Article 21. https://doi.org/10.3390/biology9010021
Silva, J. D., Yue Su, Calfee, C. S., Delucchi, K. L., Weiss, D., McAuley, D. F., O'Kane, C. and Krasnodembskaya, A. D. (2021) Mesenchymal Stromal Cell Extracellular Vesicles Rescue Mitochondrial Dysfunction and Improve Barrier Integrity in Clinically Relevant Models of ARDS. European Respiratory Journal, 58, Article ID: 2002978. https://doi.org/10.1183/13993003.02978-2020

### EXPERIMENTAL EXAMPLES

Described herein is an experiment that was performed and demonstrates the following: after mixing EVs with isolated mitochondria, the EVs stimulated aggregation of the isolated mitochondria and improve the maintenance of mitochondrial membrane potential (MMP). Thus, the EVs can improve the storage/stability of isolated mitochondria for use in mitochondrial organelle transplantation (MOT™).

### Materials and Methods

### Isolation, Primary Culture and Cryopreservation of Human Fibroblasts

Collection of human skin tissue was approved by Western IRB (Protocol #20203104). All tissue pieces were digested by 3% collagenase type 3 (Worthington Biochemical Corporation, Lakewood, NJ, USA) at 37° C. for 5 hours in a sterile stirring chamber. The liquid was transferred into a sterile 50 ml centrifuge tube and centrifuged for 5 minutes at 400 g. Supernatant was removed. The cell pellet was resuspended in 30 ml pre-warmed (37° C.) complete alpha minimum essential media (alpha MEM) (GIBCO, Carlsbad, CA, USA) containing 5% human platelet lysate (HPL) (Biological Industries, Cromwell, CT, USA) and 0.05 mg/ml gentamicin (GIBCO, Carlsbad, CA, USA). Cells was cultured at 37° C. in 5% CO2 incubator overnight. Medium and floating cells were aspirated the next day. 30 mL fresh medium was added to the flask. The flask was returned to the incubator for culture. Culture medium was refreshed every 3-4 days. When the flask reached 80% confluency, cells were sub-cultured to new flasks by 1:10 dilution. The primary fibroblasts of second and third passages were collected, re-suspended in Nutrifreez D10 cryopreservation medium (Biological Industries, Cromwell, CT, USA), aliquoted to 1 ml containing 1×10⁶ cells in cryovials, frozen and stored in liquid nitrogen.

### Fibroblast Expansion

Human primary fibroblasts were established and stored in liquid nitrogen. Human primary fibroblasts were recovered from liquid nitrogen and cultured in alpha MEM (GIBCO, Carlsbad, CA, USA) containing 5% human platelet lysate (HPL) (Mill Creek Life Sciences, Rochester, MN, USA). When cells grew to 80% confluency in a culture flask, they were digested with TrypLE expression solution (GIBCO, Carlsbad, CA, USA) and sub-cultured at 37° C. and 5% CO2.

### Mesenchymal Stromal Cell (MSC) Culture

BM-MSCs (bone marrow-MSCs) were cultured on laminin-521 coated plates in StemMACS MSC Expansion Medium (Miltenyi Biotec, Germany) at 37° C. and 5% CO₂. Laminin-521 was purchased from Biolamina Ab, Sweden and the cell culture plates were coated according to the manufacturer's instructions. For production of EVs, BM-MSCs were cultured until approximately 80% confluency and carefully washed twice with PBS. After that, serum-free Opti-MEM™ medium (GIBCO, Carlsbad, CA, USA) was added and the cells were incubated for 48 hours at 37° C. and 5% CO2. Then, the conditioned medium was collected, and centrifuged first for 5 minutes at 700 g to remove living cells and a second spin for 100 minutes at 250 g to remove cellular debris. The cells that remained were cultured for 24 hours in the complete culturing medium. Another production round was made as described above.

### Isolation of EVs

The centrifuged conditioned medium was sterile filtered and concentrated using tangential flow filtration (TFF) using a KR2i TFF system (SpectrumLabs). For that, a modified polyethersulfone (mPES) hollow fiber filter with 300 kDa membrane pore size was used (MidiKros, 370 cm² surface area, SpectrumLabs). The flow rate was set at 100 mL/min. At first, the conditioned medium was concentrated to a volume of 75 mL in the container (100 mL total volume in the system) and, then, dialyzed with 1 liter of sterile filtered PBS. After dialysis, the sample was concentrated to 30 ml and sterilized using a 0.22 μm filter. Finally, the sample was concentrated to a volume of 1-2 ml using a 10 mL 10 kDa MWCO spin filter (Amicon).

### Western Blotting

The samples were mixed with Laemmli Sample Buffer (Bio-Rad) under reducing conditions and warmed at 95° C. for 10 min. Then, 4%-12% gradient gels were used for SDS electrophoresis and the proteins were transferred to polyvinylidene fluoride membranes using iBlot 2 (Invitrogen) according to the manufacturer's instructions. The membranes were first hybridized with the antibody of interest and, then, with the corresponding HRP-conjugated secondary antibodies. For visualization, chemoluminescent HRP-substrate from Amersham Biosciences was used and the figures were made using ChemiDoc imaging system (Bio-Rad).

### Transmission Electron Microscopy

A 5 μL drop of sample was placed on a formvar and carbon coated 200-mesh copper grid. The excess solution was removed by blotting with filter paper. The sample was then directly contrasted with 2% uranyl acetate. Excess of uranyl acetate was removed by blotting on filter paper. The contrasting step was re-peated twice. Dried grids were examined by Tecnai™ G2 Spirit BioTwin transmission electron microscope (Thermo Fisher/FEI) at 80 kV with an ORIUS SC200 CCD camera and Gatan Digital Micrograph software (both from Gatan Inc.).

### Detection of Mitochondrial Genes by Polymerase Chain Reaction (PCR)

Mitochondrially encoded cytochrome c oxidase I (MT-CO1) and mitochondrially encoded NADH dehydrogenase 1 (MT-ND1) in the samples of imEVs were detected by PCR. PCR was performed in a total 25 μL volume including 2 μL of PCR Master mix (Thermofisher Scientific, Waltham, MA, USA), 0.5 μl of 100 μM forward and reverse primers and 1 μl of heat-denatured imEVs samples. The primers for MT-CO1 amplification were 5′-CTAGCAGGTGTCTCCTCTATCT-3′ (SEQ ID NO. 1) and 5′-GCTCGTGTGTCTACGTCTATTC-3′ (SEQ ID NO. 2). The primers for MT-ND1 were 5′-CCTTCGCTGACGCCATAAA-3′ (SEQ ID NO. 3) and 5′-GGTCTCTGCTAGTGTGGAGATA-3′ (SEQ ID NO. 4). β-actin (ACTB) was used as an endogenous gene control. The primers for ACTB amplification were 5′-CCTTTCTCACTGGTTCTCTCTTC-3′ (SEQ ID NO. 5) and 5′-CGTAGCACAGCTTCTCCTTAAT-3′ (SEQ ID NO. 6). The amplification procedure included initial DNA denaturing at 95° C. for 3 minutes, then 35 cycles of denaturing 30 seconds at 95° C., primer annealing 30 seconds at 50° C. and 60 seconds of extension at 72° C., and final extension of 10 minutes at 72° C. in a T100 Thermal Cycle (Bio-Rad, Hercules, CA, USA). PCR products were run on a 2% agarose gel and imaged by ethidium bromide fluorescence.

### Isolation of Mitochondria

Mitochondrial isolation follows a previously described protocol in Jiang, X., Baucom, C. and Elliott, R. (2020) Mitochondria Dynamically Transplant into Cells in Vitro and in Mice and Rescue Aerobic Respiration of Mitochondrial DNA-Depleted Motor Neuron NSC-34. Journal of Biomedical Science and Engineering, 13, 203-221. https://doi.org/10.4236/jbise.2020.139019, which is incorporated by reference in its entirety. All reagents were sterile. Fibroblasts were centrifuged for 5 minutes at 400 g and at 4° C. to remove the media. Cell pellet was re-suspended in ice-cold 300 mM sucrose mitochondrial isolation buffer (MIB) (Sigma Aldrich, St. Louis, MO, USA) and homogenized by bead beating (Bead Ruptor 12, Omni International homogenizer company, Kennesaw, GA, USA). The cell lysate was centrifuged for 10 minutes at 700 g and at 4° C. Then the supernatant was transferred to new centrifugation tubes and centrifuged for 10 minutes at 9000 g at 4° C. The supernatant was removed. The wet weight of mitochondria was measured. The mitochondrial pellet was re-suspended with 240 mM sucrose mitochondrial respiration buffer (MRB) (Sigma Aldrich, St. Louis, MO, USA). The isolated mitochondria of fibroblasts was then mixed with imEVs (9:1 in volume) and stored the mixture at 2° C.-6° C. for 2 and 5 days. MMP and mitochondrial ATP content were measured and compared to fresh mitochondria (0 day).

### Measurement of MMP

MMP generated by proton pumps is an essential component in the process of energy storage during OXPHOS. Membrane potential dependent dyes such as JC-1 (5,5′,6,6′-tetrachloro-1,1′,3,3′-tetraethylbenzimidazolocarbocyanine iodide) and MitoTracker dyes (rosamine- or cyCarbocyanine-based probes) have been used to stain mitochondria and monitor mitochondrial potential. A stock solution of JC-1 was added to mitochondrial suspension to a final concentration 1 μg/mL. The mixture was incubated for 10 minutes at room temperature. For a control assay, stock solution of valinomycin was added to mitochondrial sample to a final concentration of 10 μg/mL. The mitochondrial sample containing valino-mycin was kept on ice for 10 minutes to allow complete dissipation of the MMP.

The valinomycin-treated mitochondria were stained with JC-1 dye for 10 minutes at room temperature. Red fluorescent J-aggregates in intact mitochondria could then be observed under fluorescent microscope. The relative fluorescence units (RFU) could be read in a multiple plate fluorimeter using an end-point method with the exicitation/emission settings of Ex/Em: 490 nm/590 nm.

### Measurement of Mitochondrial ATP Content

ATP content was measured with ATPlite kit (Perkin Elmer Inc., Waltham, MA, USA). The detailed procedure used was from the product manual. In brief, the method was carried out as follows: 50 μL of mammalian cell lysis solution was added to 100 μL of mitochondria, the mixture of mitochondria with imEVs, or MRB per well in a 96-well plate with white wells and clear bottom. The plate was shaken the plate for 5 minutes. 50 μL of substrate solution was added to all wells and the plate was shaken for 5 minutes. The luminescence of the plate was then measured, allowing for the calculation of the ATP content of samples using a standard curve.

### Statistical Analysis

The student's t-test was used to test statistical significance. A p-value less than 0.05 was judged to be of statistical significance.

### Results

Isolation & Characterization of imEVs

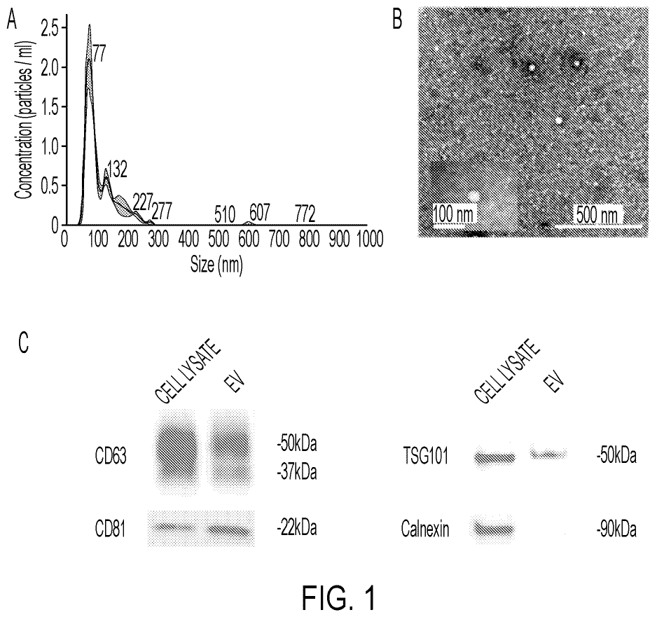

For generation of imEVs, extracellular vesicles (EV) were isolated from the cell culture medium pre-conditioned by bone marrow derived mesenchymal stromal cells (BM-MSCs) as described herein. imEVs were characterized according to the International Society for Extracellular Vesicles guidelines. The concentration and size distribution were evaluated using NanoSight 3000 device (FIG. 1, panel A). The main peak corresponds to the characteristic size for exosomes. The EVs were also characterized by transmission electron microscopy as described herein revealing characteristic sizes and shapes for exosomes (FIG. 1, panel B). The scale bar in FIG. 1, panel B is 500 nm, while the inset has a scale bar of 100 nm. Also, the EV preparation and cell lysate from the parental cells were characterized for expression of positive markers of EVs (CD63, CD81, and TSG101) and negative marker of EVs (Calnexin) using West-blotting as described below. The lysate showed reactivity to antibodies against all four markers, while the EVs showed reactivity to only three of the markers and not to Calnexin (FIG. 1, panel C).

Viable Mitochondria in imEVs

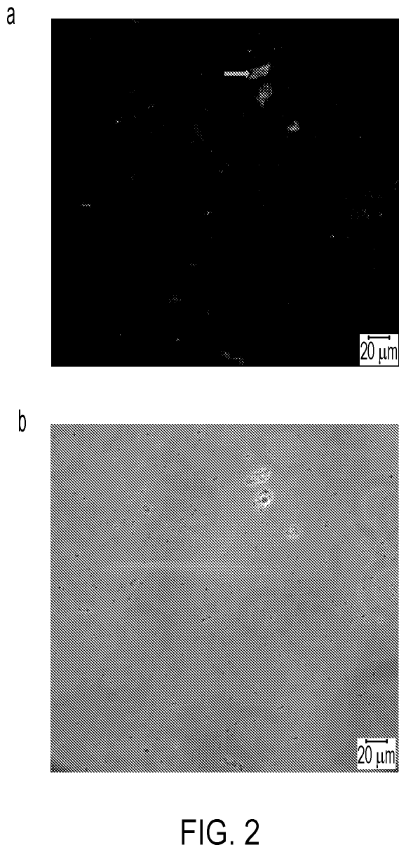

imEVs were shipped on dry ice and stored at −20° C. after arrival. At 7 days post-freezing, imEVs were thawed at 4° C. and stained with JC-1. The presence of mitochondrial genes in imEVs was determined using PCR. Intact mitochondria actively concentrated the JC-1 dye in the imEVs sample (e.g., as shown in FIG. 2). FIG. 2, panels A and B show JC-1 stained mitochondria in imEVs. FIG. 2, panel A shows a fluorescent image of the JC-1 stained mitochondria. FIG. 2, panel B shows an overlay of phase contrast and fluorescent images. Arrows point to J-aggregates of mitochondria.

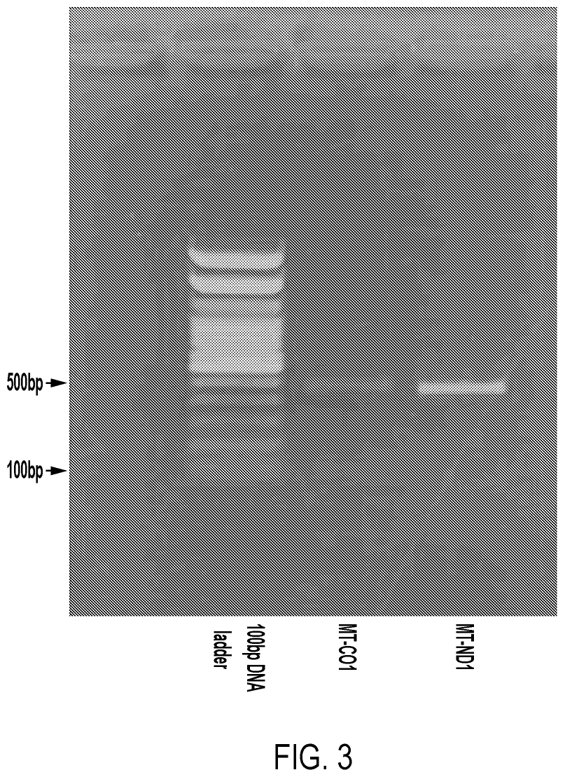

Mitochondrial genes MT-ND1 and MT-CO1 in the imEVs were amplified by PCR (e.g., as shown in FIG. 3). FIG. 3 shows a Western Blot for genes in imEVs. JC-1 staining and PCR confirmed imEVs contained viable mitochondria. The viable mitochondria maintained mitochondrial membrane potential (MMP) even though the imEVs went through freezing-thawing cycles. The viable mitochondria may be apoptotic bodies or free mitochondria in the imEVs solution. The results suggest that imEVs may enhance the stability of extracellular mitochondria.

imEVs Stabilize the Isolated Mitochondria's MMP in Cold Storage

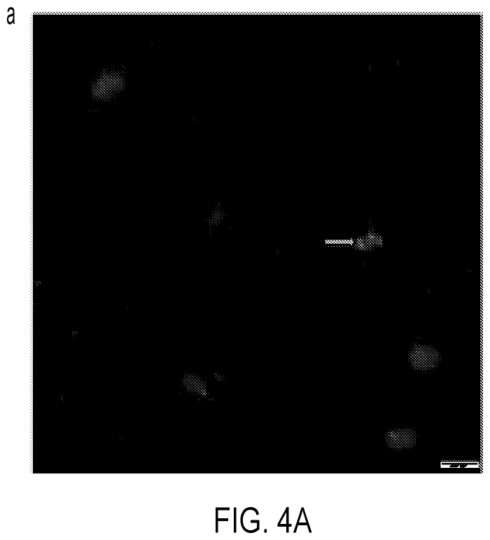

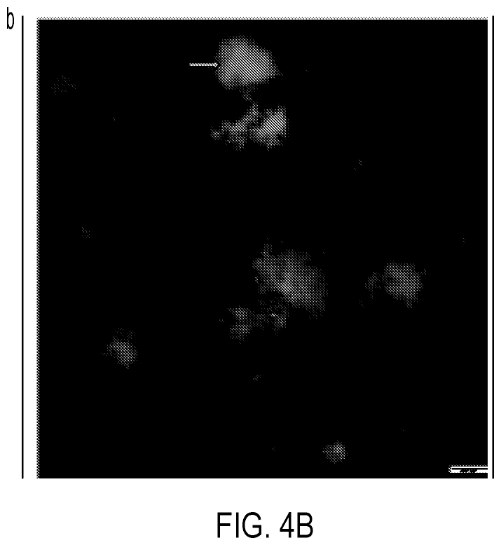

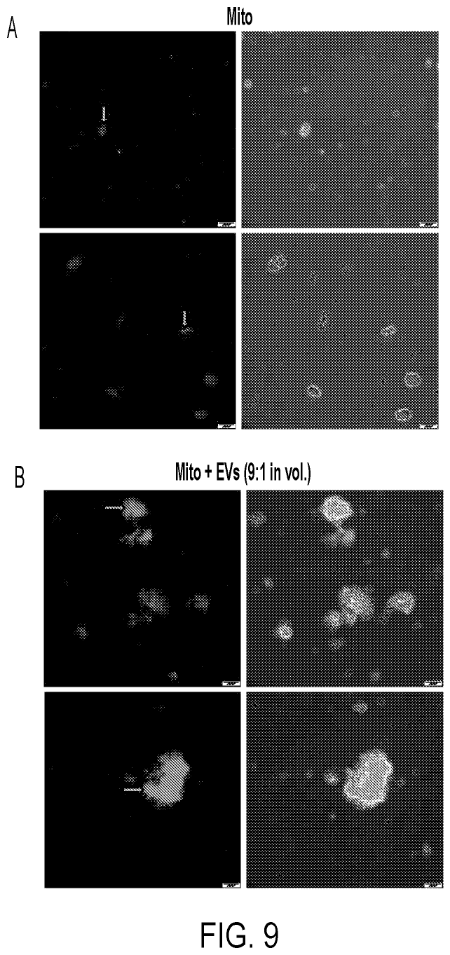

Mitochondria alone and a mixture of mitochondria with imEVs were stored at 2° C.-6° C. for 2 and 5 days. Microscopy showed that imEVs stimulated aggregation of the isolated mitochondria and, further, that the isolated mitochondria in the imEV and mitochondria mixture maintained MMP as compared to mitochondria without imEVs (e.g., as shown in FIGS. 4A and 4B, and further exemplary photos are shown in FIGS. 9A and B). FIGS. 4A and 9A show mitochondria without imEVs. FIGS. 4B and 9B show mitochondria mixed with imEVs (9:1 in volume). The arrows in FIGS. 4A and 4B and FIGS. 9A and 9B show J-aggregates of mitochondria.

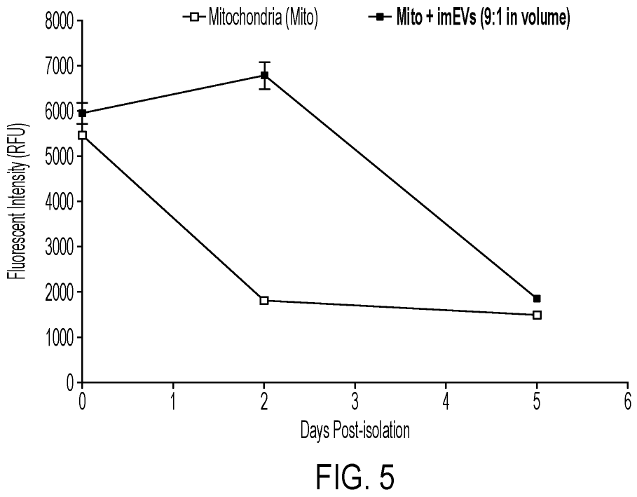

MMP was measured by a multiple plate fluorimeter using an end-point method with an excitation/emission setting of Ex/Em: 490 nm/590 nm. The MMP of isolated mitochondria and isolated mitochondria with imEVs was compared to the MMP of fresh mitochondria (“0 day”). Isolated mitochondria stored at 2° C.-6° C. for 2 days lost a significant amount of MMP (RFU 5458±52 at 0 day vs. 1822±68 at 2 days, p<0.01). The MMP at 2 days of isolated mitochondria without imEVs was approximately 33% of the MMP of freshly isolated mitochondria at 0 days as shown in Table 1 below. However, mitochondria mixed with imEVs maintained and even slightly increased the MMP after 2 days of storage at 2° C.-6° C. (RFU 5962±222 at 0 day vs. 6786±291 at 2 days, p>0.05, i.e., as seen in Table 1 below). Mitochondria with imEVs have significantly higher MMP than the mitochondria without imEVs (6786±291 vs. 1822±68, p<0.01) after 2 days of preservation at 2° C.-6° C. (Table 1). After 5 days, both the mitochondrial without and with imEVs lost 73% and 70% of MMP (p<0.01, P<0.01, compared to fresh mitochondria), respectively (i.e., as shown in Table 1 and FIG. 5). The preliminary results showed that imEVs prolonged the survival of isolated mitochondria in cold storage at least 2 days.

TABLE 1
imEVs preserved MMP of isolated mitochondria.

| | Time Post-Isolation | | |
|---|---|---|---|
| | 0 day | 2 days | 5 days |
| Mitochondria | 5458 ± 52 (3)¹ | 1822 ± 68 (3) | 1490 ± 12 (3) |
| (without imEVs) | | (p < 0.01)² | (p < 0.01)² |
| Mitochondria + | 5962 ± 222 (3) | 6786 ± 291 (3) | 1838 ± 37 (3) |
| imEVs | (p > 0.05)³ | (p > 0.05)² | (p < 0.01)² |
| (9:1 in volume) | | (p < 0.01)³ | (p > 0.05)³ |

The results from Table 1 correspond to mitochondria stained with JC-1. RFU was measured by multiple plate fluorimeter using end-point method with the setting of Ex/Em: 490 nm/590 nm. The meaning of the superscript annotations is provided as follows: ¹Relative fluorescence units (RFU), mean±standard deviation (SD) (N); ²Compared to fresh mitochondria (“0 day”); ³Compared to the Mitochondria (without imEVs).

imEVs Preserve ATP Content of the Isolated Mitochondria

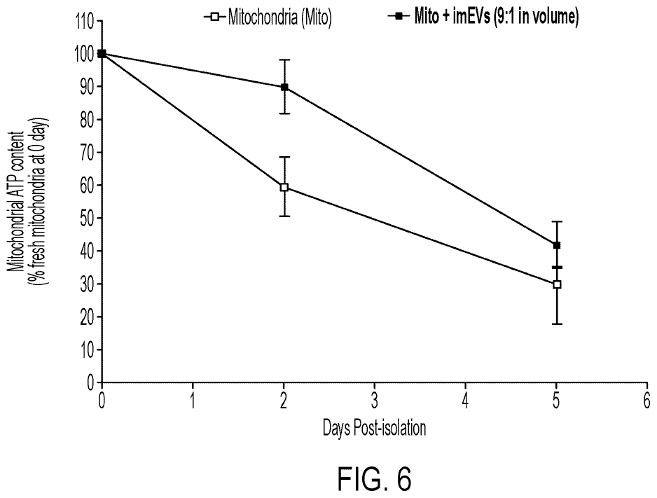

In agreement with the MMP results, isolated mitochondria lost approximately 40% ATP content after 2 days cold storage at 2° C.-6° C. as compared to freshly isolated mitochondria (“0 day”) (p<0.01). In contrast, the mitochondria mixed with imEVs maintained approximately 90% ATP content as compared to fresh mitochondria (p>0.05) (i.e., as shown in Table 2). Mitochondria with imEVs have significantly higher ATP content than the mitochondria without imEVs (90±8 vs. 60±9, p<0.05) (Table 2). After 5 days of cold storage, mitochondria in both solutions lost significant ATP content (p<0.01, p<0.01, respectively, compared to fresh mitochondria) (i.e., as shown in Table 2 and FIG. 6). In FIG. 6, mitochondrial ATP content was calculated as the percentage of the fresh isolated mitochondria.

TABLE 2
imEVs enhance the stability of mitochondrial ATP.

| | Time Post-Isolation | | |
|---|---|---|---|
| | 0 day | 2 days | 5 days |
| Mitochondria | 5458 ± 52 (3)¹ | 1822 ± 68 (3) | 1490 ± 12 (3) |
| (without imEVs) | | (p < 0.01)² | (p < 0.01)² |
| Mitochondria + | 5962 ± 222 (3) | 6786 ± 291 (3) | 1838 ± 37 (3) |
| imEVs | (p > 0.05)³ | (p > 0.05)² | (p < 0.01)² |
| (9:1 in volume) | | (p < 0.01)³ | (p > 0.05)³ |

In Table 2, mitochondrial ATP content was calculated as the percentage of the fresh isolated mitochondria. The meaning of the superscript annotations is provided as follows: ¹Relative fluorescence units (RFU), mean 1 standard deviation (SD) (N); ²Compared to fresh mitochondria (0 day); ³Compared to the Mitochondria (without imEVs).

### Discussion

In this series of experiments, it was found that EVs contain viable mitochondria after freezing and thawing. The respiratory capacity and adenosine triphosphate (ATP) production of the mitochondria may be improved as well by using EVs. Mitochondrial genes in the EV sample were amplified by PCR (polymerase chain reaction). After mixing the EVs with isolated mitochondria (for use in MOT™), the EVs stimulated aggregation of the isolated mitochondria and improve the maintenance of mitochondrial membrane potential (MMP). Thus, the EVs can improve the storage/stability of isolated mitochondria for use in MOT™.

Isolated mitochondria lose significant activity when stored on ice for more than 1 hour. Thus, clinical applications of mitochondrial transplantation would benefit from improved storage. The development of mitochondrial storage for an extended period is a very important issue. EVs are phospholipid bilayer-enclosed vesicles naturally released from all cell types. EVs carry cargos including proteins, nucleic acids, lipids, metabolites, and even organelles such as mitochondria from the parent cells. The inventors found viable mitochondria in imEVs using JC-1 staining and PCR assays, even though the imEVs had gone through freezing-thawing cycles. Without wishing to limit other notable benefits, EVs preserved the viability of mitochondria that were enclosed within the EVs themselves (apoptotic bodies) or outside of the EVs (mitochondrial contaminants). To confirm these findings, isolated mitochondria of fibroblasts was mixed with imEVs of MSCs at a 9:1 ratio and stored in mitochondrial solutions at 2° C.-6° C. After 2 days of storage, isolated mitochondria without imEVs lost significant MMP and ATP content, but mitochondria with imEVs added maintained almost the same MMP and ATP content as fresh mitochondria. Moreover, imEVs promoted mitochondrial aggregation. Without wishing to be bound to any particular theory, mitochondrial aggregation may be one of mechanism which enhances mitochondrial viability. Furthermore, the results suggest that EVs can extend the viability of mitochondria in cold storage at least 2 days.

### REFERENCES

Jiang, X., Baucom, C. and Elliott, R. (2020) Mitochondria Dynamically Transplant into Cells in Vitro and in Mice and Rescue Aerobic Respiration of Mitochondrial DNA-Depleted Motor Neuron NSC-34. Journal of Biomedical Science and Engineering, 13, 203-221. https://doi.org/10.4236/jbise.2020.139019
Sivandzade, F., Bhalerao, A. and Cucullo, L. (2019) Analysis of the Mitochondrial Membrane Potential Using the Cationic JC-1 Dyeas a Sensitive Fluorescent Probe. Bio Protocol, 9, e3128. https://doi.org/10.21769/BioProtoc.3128
Elsharkasy, O. M., Nordin, J. Z., Hagey, D. W., de Jong, O. G., Schiffelers, R. M., Andaloussi, S. E. and Vader, P. (2020) Extracellular Vesicles as Drug Delivery Systems: Why and How? Advanced Drug Delivery Reviews, 159, 332-343. https://doi.org/10.1016/j.addr.2020.04.004.

### Supplemental Data

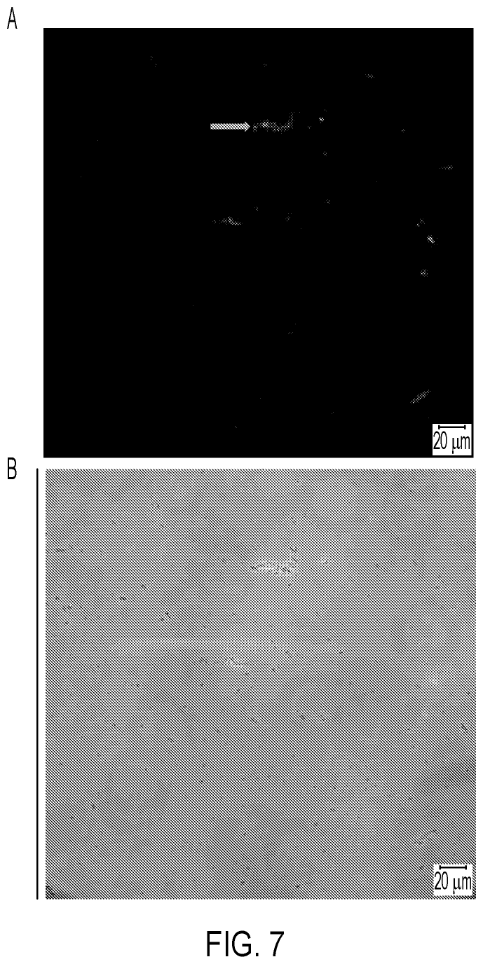

FIG. 7, panels A and B are images showing stained mitochondria in mixtures of extracellular vesicles (EVs). In FIG. 7, panels A and B, the mitochondria are shown in red with arrows drawing attention to mitochondria within structures formed by the EVs. The source of the EVs were mesenchymal stromal cells (MSCs). The EVs arrived frozen and were stored at −20° C. then kept at 4° C. after thawing. The legend at bottom right in both panels A and B depicts a 20 micrometer length.

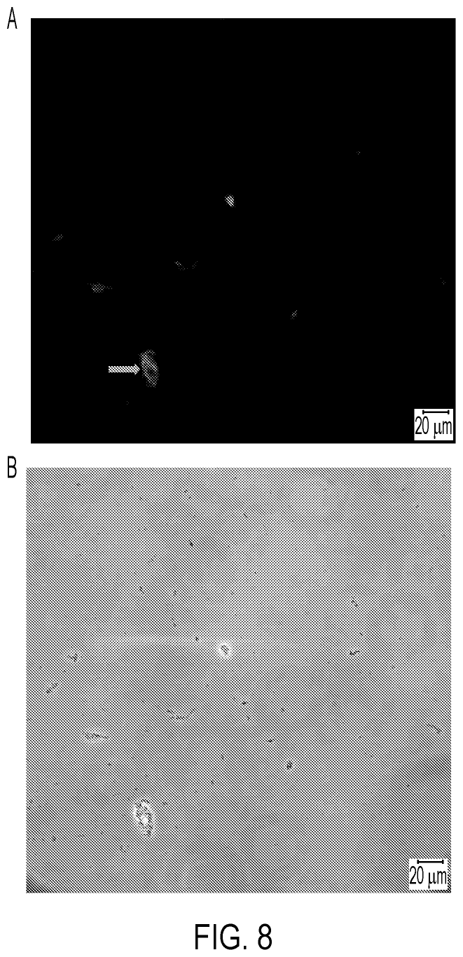

FIG. 8, panels A and B are images showing stained mitochondria in mixtures of extracellular vesicles (EVs). The mitochondria in FIG. 8, panels A and B are shown in red, with arrows drawing attention to mitochondria within structures formed by the EVs. The source of the EVs was mesenchymal stromal cells (MSCs). The EVs arrived frozen and were stored at −20° C. then kept at 4° C. after thawing. The legend at bottom right depicts a 20 micrometer length.

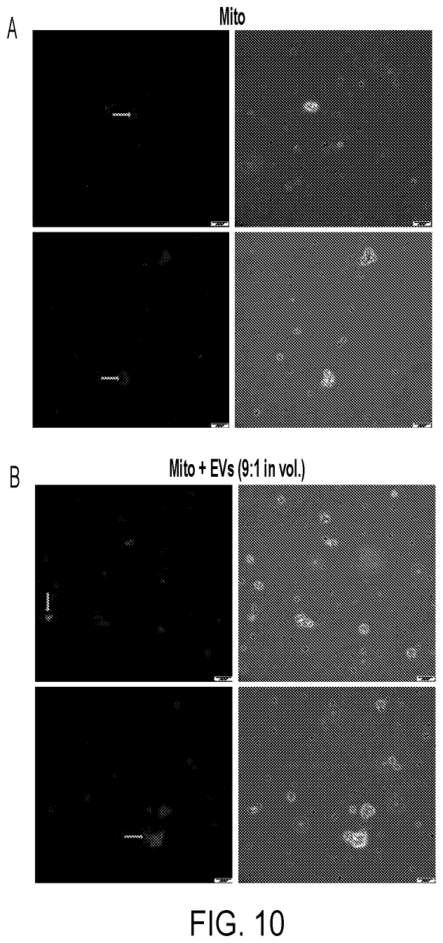

FIG. 10 depicts mitochondrial membrane potential 5 days post-isolation and also shows that EVs help maintain mitochondrial aggregation and improve the maintenance of MMP at the 5 day mark. The upper series of 4 images under the title “Mito” shows isolated mitochondria without EVs. The lower series of 4 images under the title “Mito+EVs (9:1 in vol.) shows mitochondria mixed with EVs.

### EXEMPLARY EMBODIMENT

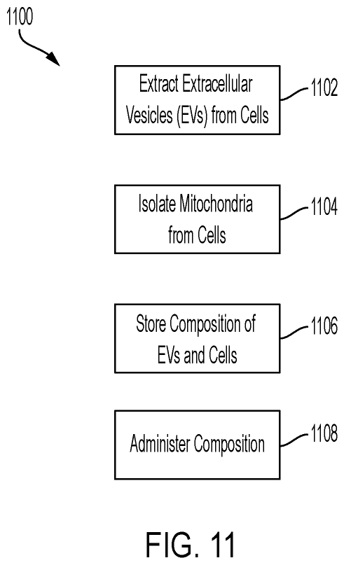

FIG. 11 is an illustrative embodiment of an exemplary method (1100) according to aspects of the present embodiments.

In some embodiments, extracellular vesicles (EVs) are extracted from cells (1102) as described herein and, for example, in section B. For example, in certain embodiments, EVs (e.g., imEVs) are obtained from MSCs cultured on a substrate (e.g., a cell culture plate, e.g., a glass cell culture plate, a plastic cell culture plate) coated with a protein (e.g., an extracellular matrix protein). In certain embodiments, cells are cultured on a substrate coated with laminin (e.g., laminin-521). In certain embodiments, EVs are positive for one, two, or all three of the following surface markers: CD63, CD81, and TSG101. In certain embodiments, EVs are negative for the marker Calnexin.

In some embodiments, mitochondria are isolated from cells (e.g., cells of a donor) (1104). For example, mitochondrial isolation methods are described herein (e.g., in sections A and C) and are described in International (PCT) Patent Application No. PCT/US2020/047359, filed Aug. 21, 2020, which is incorporated by reference in its entirety. In some embodiments, isolated mitochondria comprises mitochondria isolated from a human donor. In certain embodiments, isolated mitochondria are obtained from fibroblasts (e.g., fibroblasts of a human donor, e.g., primary fibroblasts of a human donor) or mesenchymal stromal cells (MSCs) (e.g., MSCs of a human donor).

In some embodiments, isolated mitochondria and EVs (e.g., imEVs) are combined in a composition to improve preservation of mitochondrial membrane potential (MMP) of the isolated mitochondria and/or improve preservation/retention of mitochondrial adenosine triphosphate (ATP) content, as described herein, for example, in section B. In certain embodiments, mitochondria accumulate within structures formed by EVs in a composition. In certain embodiments, a mixture of mitochondria and EVs has a ratio from about 1:50 (mitochondria:Evs, in vol.) to about 50:1 (mitochondria:Evs, in vol.). In certain embodiments, the ratio is from about 2:1 to about 50:1. In certain embodiments, the ratio is from about 5:1 to about 15:1. In certain embodiments, the ratio is about 9:1. In some embodiments, compositions are or contain storage or isolation buffers as described herein, for example, in section C and in International (PCT) Patent Application No. PCT/US2020/047359, filed Aug. 21, 2020, which is incorporated by reference in its entirety.

In some embodiments, a composition (e.g., a storage composition) comprising mitochondria and EVs is stored (1106) (e.g., preserved) at a reduced temperature as described herein, for example, in section B. As described herein, a composition is stored at a temperature below 15° C., e.g., below 10° C., e.g., at a temperature within a range from about 0° C. to about 15° C., e.g., from about 1° C. to about 10° C., e.g., from about 2° C. to about 6° C.). In certain embodiments, compositions comprising EVs and mitochondria are stored for a time up to about 24 hours, e.g., up to about 48 hours, e.g., up to about 5 days). In certain embodiments, compositions comprising EVs and mitochondria are stored for at least 1 hour (e.g., at least 2 hours, at least 3 hours, at least about 6 hours, at least about 12 hours, at least about 24 hours, at least about 48 hours, at least about 5 days). In some embodiments, storage (e.g., preservation) of a composition preserves MMP and/or ATP content of the isolated mitochondria as described herein, for example in section B. In certain embodiments, compositions described herein are stored at or below 0° C. (e.g., at or below −10° C., at or below −20° C.). For example, compositions can be stored using liquid nitrogen or on dry ice.

In some embodiments, after a storing step, a composition comprising stored mitochondria (e.g., a mitochondrial composition) (e.g., a pharmaceutical composition) is administered to a subject (1108), for example, as part of a method of mitochondrial organelle transplantation (MOT). For example, in some embodiments, compositions comprising mitochondria for use in administration to a subject are described herein, for example, in section C and International (PCT) Patent Application No. PCT/US2020/047359, filed Aug. 21, 2020, which is incorporated by reference in its entirety. In some embodiments, a composition comprising the stored mitochondria is administered to a subject who has a mitochondrial dysfunction or other condition (e.g., an injury, a cardiac disease) as described herein, for example, in section A and International (PCT) Patent Application No. PCT/US2020/047359, filed Aug. 21, 2020, U.S. Provisional Patent Application No. 63/405,336, filed Sep. 9, 2022, and International (PCT) Patent Application No. PCT/US2020/047359, filed Aug. 21, 2020, each of which are incorporated by reference in their entireties.

It should be understood that the order of steps or order for performing certain action is immaterial so long as operability is not lost. Moreover, two or more steps or actions may be conducted simultaneously.

It is contemplated that compositions, methods, kits, and processes of the claimed invention encompass variations and adaptations developed using information from the embodiments described herein. Adaptation and/or modification of the compositions, methods, and processes described herein may be performed, as contemplated by this description.

While the invention has been particularly shown and described with reference to specific preferred embodiments, it should be understood by those skilled in the art that various changes in form and detail may be made therein without departing from the spirit and scope of the invention as defined by the appended claims.

LISTING OF SEQUENCES
SEQ ID NO. 1
CTAGCAGGTGTCTCCTCTATCT
SEQ ID NO. 2
GCTCGTGTGTCTACGTCTATTC
SEQ ID NO. 3
CCTTCGCTGACGCCATAAA
SEQ ID NO. 4
GGTCTCTGCTAGTGTGGAGATA
SEQ ID NO. 5
CCTTTCTCACTGGTTCTCTCTTC
SEQ ID NO. 6
CGTAGCACAGCTTCTCCTTAAT

What is claimed is:

1. A composition, the composition comprising:
isolated mitochondria; and
extracellular vesicles (EVs).

2. The composition of claim 1, wherein the extracellular vesicles comprise one or more members selected from the group consisting of (i), (ii), and (iii) as follows:
(i) microvesicles (MVs), exosomes, and apoptotic bodies;
(ii) microvesicles (MVs); and
(iii) apoptotic bodies.

3. The composition of claim 1, wherein the extracellular vesicles comprise microvesicles (MVs).

4. The composition of claim 3, wherein the microvesicles (MVs) range from about 100 nm to about 1 micrometer in diameter.

5. The composition of claim 3, wherein the microvesicles (MVs) comprise cytosolic and plasma membrane associated proteins.

6. The composition of claim 1, wherein the extracellular vesicles (EVs) comprise microvesicles (MVs).

7. The composition of claim 6, wherein the microvesicles (MVs) range from about 30 nm to about 150 nm in diameter.

8. The composition of claim 6, wherein the microvesicles (MVs) are formed by an endosomal route.

9. The composition of claim 1, wherein the extracellular vesicles comprise apoptotic bodies.

10. The composition of claim 9, wherein the apoptotic bodies range from about 50 nm up to about 5 micrometers in diameter.

11. The composition of claim 9, wherein the apoptotic bodies comprise one or more members selected from the group consisting of intact organelles, chromatin, and glycosylated proteins.

12. The composition of claim 1, wherein the extracellular vesicles comprise extracellular vesicles of mesenchymal stromal cells (imEVs).

13. The composition of claim 12, wherein the imEVs are obtained from mesenchymal stromal cells cultured on a substrate coated with laminin.

14. The composition of claim 1, wherein the isolated mitochondria comprises mitochondria isolated from fibroblasts.

15. The composition of claim 1, wherein the isolated mitochondria comprises mitochondria isolated from mesenchymal stromal cells (MSCs).

16. The composition of claim 1, wherein the composition comprises a mixture of mitochondria and EVs in a ratio from about 1:50 (mitochondria:EVs, in vol.) to about 50:1 (mitochondria:EVs, in vol.).

17. The composition of claim 1, wherein the composition comprises a mixture of mitochondria and EVs in a ratio from about 5:1 (mitochondria:EVs, in vol.) to about 15:1 (mitochondria:EVs, in vol.).

18. The composition of claim 1, wherein the composition is or comprises a mitochondrial storing buffer.

19. The composition of claim 18, wherein the mitochondrial storing buffer comprises:
a buffering agent;
a chelating agent;
a sugar;
an agent that acts as a membrane stabilizer and/or oxygen radical scavenger and/or binder of Ca²⁺ and/or binder of free fatty acid; and
a serine protease inhibitor.

20. The composition of claim 1, wherein the composition is or comprises a mitochondrial isolation buffer composition.

21. The mitochondrial isolation buffer composition of claim 20, wherein the mitochondrial isolation buffer composition comprises:
a buffering agent;
a chelating agent;
a sugar;
an agent that acts as a membrane stabilizer and/or oxygen radical scavenger and/or binder of Ca²⁺ and/or binder of free fatty acid (e.g., bovine serum albumin, BSA); and
a serine protease inhibitor.

22. The composition of claim 1, wherein the composition comprises a pharmaceutically acceptable carrier.

23. The composition of claim 1, wherein the mitochondria accumulate within structures formed by the EVs in the composition.

24. The composition of claim 1, wherein the composition does not comprise an antibiotic.

25. The composition of claim 1, wherein the EVs are positive for one, two, or all three of the following surface markers: CD63, CD81, and TSG101.

26. The composition of claim 1, wherein the EVs are negative for the surface marker Calnexin.

27. The composition of claim 1, wherein the composition can be stored for a time up to about 5 days (e.g., up to about 24 hours, e.g., up to about 48 hours) (e.g., without a significant loss of mitochondrial membrane potential (MMP)).

28. The composition of claim 1, wherein the mitochondrial ATP content of the isolated mitochondria is at least 80% of the mitochondrial ATP content of freshly isolated mitochondria.

29. The composition of claim 1, wherein the mitochondrial membrane potential (MMP) of the isolated mitochondria is substantially the same as the MMP of freshly isolated mitochondria.

30. The composition of claim 1, wherein the composition can be stored for at least about 48 hours or more.

31. The composition of claim 1, wherein the composition can be stored at a reduced temperature.

32. The composition of claim 31, wherein the reduced temperature is a temperature below 15° C.

33. The composition of claim 31, wherein the reduced temperature is a temperature at or below 0° C.

34. A method comprising using a composition comprising isolated mitochondria and extracellular vesicles to improve preservation of mitochondrial membrane potential (MMP) of the isolated mitochondria, wherein the isolated mitochondria are used in any of the mitochondrial organelle transplantation methods and/or compositions and/or kits described in the above-referenced International (PCT) Patent Application No. PCT/US2020/047359 or U.S. Provisional Patent Application No. 63/405,336, both incorporated herein by reference.

35. A method for transplantation of mitochondria in a human subject, said method comprising:
storing the mitochondria isolated from a donor in a storage composition comprising extracellular vesicles (EVs); and
after the storing step, administering to said subject a mitochondrial composition comprising said stored mitochondria.

36. The method of claim 35, wherein the method comprises storing the storage composition at a reduced temperature.

37. The method of claim 36, wherein the reduced temperature is a temperature below 15° C.

38. The method of claim 36, wherein the reduced temperature is a temperature at or below 0° C.

39. The method of claim 35, wherein the method comprises storing the storage composition for at least about 48 hours or more prior to administering the composition.

40. The method of claim 35, wherein the extracellular vesicles comprise one or more members selected from the group consisting of (i), (ii), and (iii) as follows: (i) microvesicles (MVs), exosomes, and apoptotic bodies:
(ii) microvesicles (MVs); and
(iii) apoptotic bodies.

41. The method of claim 35, wherein the extracellular vesicles comprise extracellular vesicles of mesenchymal stromal cells (imEVs).

42. The method of claim 35, wherein the subject has a neurodegenerative disease or other condition associated with mitochondrial dysfunction.

43. The method of claim 35, wherein the mitochondrial composition further comprises a mitochondrial storing buffer having a potassium ion concentration safe for administration to humans.

44. The method of claim 35, wherein the administering step comprises parenterally administering at least one-unit dose of said mitochondrial composition to said subject.

45. The method of claim 35, wherein the administering step comprises both intramuscular injection and intravenous injection of said mitochondrial composition to said subject.

46. The method of claim 35, wherein the mitochondrial composition administered to the subject does not comprise an antibiotic.

47. The method of claim 35, comprising administering to the subject an iron-chelating agent.

48. The method of claim 35, comprising administering to the subject an antioxidant and/or a probiotic.

49. A kit comprising a mitochondrial composition in a unit dosage effective to treat a neurodegenerative disease or other condition associated with mitochondrial dysfunction in a subject, said mitochondrial composition comprising:
isolated mitochondria; and
extracellular vesicles (EVs).

50. The kit of claim 49, further comprising instructions for optimizing the dose and/or frequency and/or route of administration of the composition.

51. A method of storing a composition comprising isolated mitochondria and extracellular vesicles to improve preservation of MMP of the isolated mitochondria and/or to improve preservation/retention of mitochondrial adenosine triphosphate (ATP) content, the method comprising storing said composition at a reduced temperature.

52. The method of claim 51, wherein the reduced temperature is a temperature below 15° C.

53. The method of claim 51, wherein the reduced temperature is a temperature at or below 0° C.

54. The method of claim 51, wherein the method comprises storing the composition for a time up to about 24 hours.

55. The method of claim 51, wherein the method comprises storing the composition for at least one hour.

56. The method of claim 51, wherein the mitochondrial ATP content of the isolated mitochondria is at least 80% of the mitochondrial ATP content of freshly isolated mitochondria.

57. The method of claim 51, wherein the mitochondrial membrane potential (MMP) of the isolated mitochondria is substantially the same as the MMP of freshly isolated mitochondria.
